(19) United States
(12) Patent Application Publication
(10) Pub. No.: US 2018/0193426 A1
(43) Pub. Date: Jul. 12, 2018

(54) INSULINS COMPATIBLE WITH NEW GENERATION IMPLANTABLE PUMPS

(71) Applicant: PhysioLogic Devices, Inc., Alpine, CA (US)

(72) Inventors: Peter C. Lord, Alpine, CA (US); William Van Antwerp, Valencia, CA (US)

(73) Assignee: PhysioLogic Devices, Inc., Alpine, CA (US)

(21) Appl. No.: 15/808,814

(22) Filed: Nov. 9, 2017

Related U.S. Application Data
(60) Provisional application No. 62/419,758, filed on Nov. 9, 2016.

(51) Int. Cl. A61K 38/28; A61K 9/00; A61M 31/00

(52) U.S. Cl. CPC A61K 38/28; A61K 9/0019; A61M 2025/0057; A61M 2205/7581; A61M 2210/1017; A61M 31/002

(57) ABSTRACT

Exemplary embodiments of the present invention are directed to stabilized insulin compositions suitable for use in implantable artificial pancreatic devices. In embodiments, purified insulin may be made more stable by introducing an insulin analog that will form a heterodimer with the purified insulin. For example, a quantity of between ½ to 10% of one of A21 desamido insulin or Lispro may be added to a purified insulin preparation, resulting in a more stable insulin. Various other methods and preparations of stable insulins, and various catheters for delivering insulins from an implantable device to a body are also presented.

### CROSS-REFERENCE TO RELATED APPLICATIONS

The present application claims the benefit of U.S. Provisional Patent Application No. 62/419,758, filed on Nov. 9, 2016, the disclosure of which is incorporated herein as if fully set forth.

### TECHNICAL FIELD

Embodiments of the present invention relate generally to implantable artificial pancreatic devices (implantable devices that measure glucose levels and automatically dispense insulin, of various types), and in particular to novel formulations of stabilized insulin that are compatible with, and may be used in optimizing, such devices.

### BACKGROUND OF THE INVENTION

In healthy individuals the pancreas excretes a small amount of insulin as a basal supply, and larger amounts as blood glucose increases after meals. This induces the blood glucose levels to fall to normal (e.g., 5 mmol/l). However, normal secretion of insulin in diabetic, but otherwise healthy, individuals may be achieved by means of artificial infusion systems. These include fully implantable closed loop insulin pumps, driven by algorithms responding to various sensors, as well as external infusion pumps worn on the shoulder, though, or similar anatomical area. During the development of these systems however, a severe problem emerged. Insulin has a tendency to denature, and aggregate, leading to precipitation. In external infusion pumps, catheter blockage can be a significant source of clinical complication, and the infusion of altered insulin has been seen as the cause of adverse effects.

With fully implantable pumps, the associated difficulties are even greater, because residence times in the reservoirs may be relatively long, thermal exposure is greater (the pump reservoir), and there may be long term contact with hydrophobic surfaces such as silicone rubber. This exacerbates the problem.

### BRIEF DESCRIPTION OF THE DRAWINGS

FIG. 1 depicts an exemplary catheter enhanced to deliver insulin in accordance with various embodiments.

FIG. 2A depicts exemplary methods to absorb phenol in silicone rubber used to manufacture an exemplary catheter, by increasing a surface to volume ratio at a tip in accordance with various embodiments.

FIG. 2B illustrates possible locations for absorbent material for either phenol, or catalyst for phenol breakdown in a catheter fluid path, in accordance with various embodiments.

FIG. 3 depicts an exemplary method to remove zinc by reduction at a catheter tip in accordance with various embodiments.

FIGS. 4A through 4E depict various embodiments for heating (or both vibrating and heating) the contents of a catheter at its tip, in accordance with various embodiments.

FIG. 4A depicts a resistive helical heater, in accordance with various embodiments.

FIG. 4B depicts a warm radioisotope heater, in accordance with various embodiments.

FIG. 4C depicts use of LEDs to illuminate and heat absorptive tubing or pigment, in accordance with various embodiments.

FIG. 4D depicts an induction coil and conductive particles in the lumen, in accordance with various embodiments.

FIG. 4E depicts a piezo tube that vibrates against a passive tube, generating heat and vibration, in accordance with various embodiments.

### DETAILED DESCRIPTION OF THE INVENTION

Various embodiments relate generally to implantable artificial pancreatic devices (implantable devices that measure glucose levels and automatically dispense insulin, of various types), and in particular to novel formulations of stabilized insulin that are compatible with, and may be used in optimizing, such devices. In accordance with various embodiments, insulins are described for (i) extended storage and use in external pumps, implantable pumps and patch pumps, (ii) concentrated for use in miniaturized external pumps, implantable pumps, and patch pumps, and (iii) suitable for unrefrigerated storage to facilitate distribution and for use in populations where refrigeration is not readily available. Finally, various embodiments also relate to ways of reducing time to peak concentration of insulin in blood.

In healthy individuals the pancreas excretes a small amount of insulin as a basal supply, and larger amounts as blood glucose increases after meals. This induces the blood glucose levels to fall to normal (5 mmol/l). However, normal secretion of insulin in diabetic, but otherwise healthy, individuals can be achieved by means of artificial infusion systems. These may include fully implantable closed loop insulin pumps, driven by algorithms responding to various sensors, as well as external infusion pumps worn on the shoulder, thigh, or similar anatomical area. In embodiments, these may further include external pumps and patch pumps on the arms, legs or torso, or, for example, implantable pumps implanted in the abdomenal region, the upper buttocks, or a pacemaker site (shoulder area just under the collarbone). During the development of these systems a severe problem emerged. Insulin has a tendency to denature, and aggregate, leading to precipitation. In external infusion pumps, catheter blockage can be a significant source of clinical complication, and the infusion of altered insulin has been seen as the cause of adverse effects.

With fully implantable pumps, the associated difficulties are even greater, because residence times in the reservoirs may be relatively long, thermal exposure is greater (e.g., in the pump reservoir), and there is long term contact with hydrophobic surfaces such as, for example, silicone rubber. To overcome these problems, various insulin preparations have been proposed and used. One of these has been Hoechst's HOE 21 PH, which has insulin stabilized by the addition of the surface-active polyethelyenepolypropylene glycol.

In embodiments, new generations of implantable pumps are designed to be significantly smaller, and to have longer times between insulin refills in their reservoirs than earlier versions, which were generally too large for patient comfort. A large portion of an implantable pump's size is due to its reservoir, and thus meaningful size reduction requires significantly smaller reservoirs. Thus, they require insulins that are both more concentrated, as well as physically and chemically stable for longer time intervals inside the reservoir at body temperatures.

In embodiments, new formulations of insulin that are stable for longer time periods in a reservoir, and more concentrated, may be provided, that overcome the problems with prior art insulins.

Thus, various embodiments of the present invention are directed to stabilized insulin compositions suitable for use in implantable artificial pancreatic devices. In one embodiment, purified insulin may be made more stable (and thus longer lasting) by introducing an insulin analog that will form a hetero dimer with the purified insulin. For example, a quantity of between ½ to 10% A21 desamido insulin can be added to a purified insulin preparation, resulting in a more stable insulin. In some embodiments, between 5-10% of A21 desamido insulin may be added to a purified insulin preparation. Various other methods and preparations of stable insulins are also presented. Or, for example, the stabilizing additive may be Lispro, added in between ½% to 10%, and preferably between 5% to 10% to a purified insulin preparation, resulting in a more stable insulin. Insulin lispro (marketed by Eli Lilly and Company as Humalog™) is a fast acting insulin analog. Engineered through recombinant DNA technology, the penultimate lysine and proline residues on the C-terminal end of the B-chain are reversed, hence its name. This modification does not alter receptor binding, but does block the formation of insulin dimers and hexamers. This allows larger amounts of active monomeric insulin to be immediately available for postprandial injections. Insulin lispro has one primary advantage over regular insulin for postprandial glucose control. It has a shortened delay of onset, allowing slightly more flexibility than regular insulin, which requires a longer waiting period before starting a meal after injection.

Additionally, in embodiments, insulin in the reservoir may be encapsulated in technospheres, such as, for example, fumaryl diketopiperazine (FDKP), so as to decrease time to peak, and thereby achieve a closed loop system that functions equivalently to a healthy human pancreas.

In embodiments, modifications to catheters to be used in implantable artificial pancreas devices are also presented. These may be used, inter alia, to remove zinc and phenols, as well as to heat the insulin as it is ejected into the intraperitoneal space (IPS). Thus, although certain additives may be present in the insulin within the reservoir, which may be used for insulin stability, such as, for example, zinc, or, for example, phenol or meta-cresol, for preventing bacterial growth and thus acting as a preservative, these additives may be removed at the point of infusion by such modifications to the discharge catheter. Thus, in embodiments, the insulin that is stored in a reservoir may be optimized for stability with such additives, and at the same time the insulin that reaches the patient, after removal of such additives, may be optimized for permeability and improved tissue compatibility, and may therefore have a shorter time to peak, as well as rapid clearance once discharged.

In what follows, various formulations of insulin are presented, all of which are optimized for use in, for example, implantable artificial pancreas devices, or the like. It is envisioned that such an implantable artificial pancreas may include a MEMS pump, as well as a reservoir. It is also envisioned that such devices may be as miniaturized as possible. One factor that drives the overall size of such an implantable device is the required volume of the reservoir of insulin; the constraint here is that the reservoir should hold sufficient insulin so as to not need to be refilled for many months. Thus, insulins that are both concentrated and remain effective for longer time intervals are required. In embodiments, such a combination of features allows for minimizing the size of a reservoir yet maximizing the time intervals between refills of that reservoir.

Such optimal insulins should, of course, present no incompatibility issues with the MEMS pumps that are used in such insulin pumps.

In embodiments, innovative formulations of insulin that are suitable for concentrations in the U400 to U1000+ range may be formulated. These insulins may, for example, be physically and chemically stable for at least 100 days in a pump reservoir with small amounts of air and agitation at 37° C. and can also be resistant to aggregation. Thus, such exemplary insulins (i) will not interfere with the valves and operating parts of the pump and (ii) do not form aggregates with negative consequences or trigger antibody formation when they are injected into the intraperitoneal space. These formulations are described below.

Additionally, various modifications to catheters to be used in implantable artificial pancreas devices are also presented. In embodiments, these may be used, inter alia, to remove zinc and phenols, as well as to heat the insulin as it is ejected into the IPS. Thus, although certain additives may be present in the insulin within the reservoir, they may be removed at the point of infusion by such modifications to the discharge catheter, and thus the insulin that reaches the patient may be optimized for chemical/thermal stability and physical stability, patient use, and may have a faster time to peak making it suitable for closed loop delivery. In what follows, these novel insulins and catheter modifications are described in detail.

Pump Compatible Insulin

In embodiments, a new formulation of insulin may be used that is intended to prevent corrosion of the silicon surfaces in a MEMS pump caused by the ingredients of conventional insulins. In embodiments, such novel formulations may minimize degradative interactions between concentrated insulin and MEMS pumps for use in implantable intraperitoneal insulin delivery.

It is noted that the preferred material for a small implantable pump mechanism is silicon. It is generally known that silicon corrodes slowly, approximately 35 nm per year, in alkaline solutions at pH 7.4 with the presence of cations such as Cl⁻ and PO₄⁻². (Rogers, et al., “Mechanisms for Hydrolysis of Silicon Nanomembranes as Used in Bioresorbable Electronics” Adv. Mater. 2015, 27, 1857-1864. All commercially available insulins, including Sanofi U400 Insuman™, which is labeled for intraperitoneal insulin delivery, are manufactured to be nominally alkaline with pH 7.4, so as to match the pH of the tissue into which they are injected. It is noted that they often contain NaCl for isotonicity. While the slow corrosion rate of 35 nm per year is not a problem for short term exposure, in long term applications such as implanted sensors, retinal implants and implantable pump mechanisms with thin structural elements, this corrosion, albeit slow, can significantly limit the lifetime of a new generation implantable device.

It is noted that some approaches to slow the corrosion process in a tissue environment include boron doping and anodic protection, as well as a variety of coatings including SiN, TOx, and DLC (referring to a diamond-like carbon coating, which is a nanocomposite coating that has unique properties of natural diamond low friction, high hardness, and high corrosion resistance). Organic materials such as parylene have been used to protect silicon as well. As a general rule however, such coatings are not sufficient to provide the degree of corrosion resistance for a long term, Class 3 medical device, such as an implantable insulin pump. As a result, a better solution is required.

It is noted that for implantable pumps, the challenge is medication compatibility, not tissue compatibility. This opens the possibility that the medication (insulin or insulin analog) can be formulated to be less corrosive to silicon.

In embodiments of the present invention, an insulin composition that is non-corrosive to silicon pumps can be used. This represents a different approach to the prior attempts to change the surface of the silicon. In embodiments, the primary change to the insulin formulation may be to reduce the OH⁻ concentration by buffering, to a reduced pH in the range of 6.0 to 7.0. It is here noted that chemical reaction rates generally occur at a rate directly proportional to the concentration of reactant concentration. Thus, for conventional insulins that are buffered at body pH of 7.4, a reduction of pH to 6.4 would be a factor of ten reduction in OH⁻ concentration, and a proportionate reduction in corrosion rate. In some embodiments, the tris buffer may be used for this purpose. “Tris” as used herein refers to tris(hydroxymethyl)aminomethane, or THAM, an organic compound with the formula (HOCH₂)₃CNH₂. It is extensively used in biochemistry and molecular biology. In biochemistry, tris is widely used as a component of buffer solutions, such as in TAE and TBE buffer, especially for solutions of nucleic acids. Using tris thus avoids the use of Cl— for tonicity and PO4- for buffering, which is beneficial in that Cl— and PO4- are both known to aggravate silicon corrosion. More importantly, tris has the largest temperature coefficient of pH of any buffer that is suitable for insulin. Thus, when the insulin is stored in the refrigerator the pH will be low, which, in embodiments, increases the chemical stability of the insulin during shelf storage.

In some embodiments, for example, Thermalin single chain insulin, which is very chemically stable and will therefore tolerate a lower pH, may be used at a pH of between 6.5 to 7.0, to reduce long term corrosion.

It is noted that Cl— concentration is also a factor in the corrosion of silicon. It is further noted that 0.1M NaCl is generally used in the formulation of medications because NaCl is already a constituent of all body fluids at that concentration. However, in embodiments, Br— —or any other endogenous cation—may be used as a substitute.

Alternatively, and preferably, assuming that there is no effect on the insulin, organic materials may also be used to maintain isotonicity. This would eliminate the effect of Cl⁻. Thus, in embodiments, glycerin may be used to maintain a desired tonicity, thus obviating the need for any silicon reactive anions or cations.

Moreover, as described in greater detail below, in alternate embodiments, glycerin may be used to create a significantly hypertonic insulin. In fact, in such embodiments, one may increase the glycerin to 30 milligrams per ml. If this insulin is delivered via a catheter that includes a water-permeable membrane, the catheter may suck up water from its surroundings, and create osmotic pressure in the catheter. Thus, in embodiments, by diluting the insulin as it comes out of the catheter, one generates sufficient osmotic pressure to drive the drug out of a blocked catheter tip. This effectively creates an osmotic wall on the catheter.

It is further noted that dilution will also favor insulin hexamer disassociation into monomer, thus increasing the tissue diffusion rate and shortening the insulin time to peak.

Consideration of the relationship of solubility versus pH for regular human insulin shows that as pH is lowered toward the isoelectric point, 5.3, solubility drops. Thus, in embodiments, the desire for lower pH must be balanced with the solubility of insulin at very high concentration with a TRIS buffer. Although low pH will favor reduced silicon corrosion, the pH should not be so low so as to prevent solubilization of insulin at U1000 concentration, even during refrigeration where the pH is reduced (using a temp sensitive TRIS buffer). Thus, in embodiments, a preferred value of human insulin pH, for use with U1000 concentration, may be 6.8. It is here noted that a preferred pH for analog U1000 insulin will be different than that for human insulin.

Novel Insulins with Greater Stabilities

In embodiments, insulins having an equivalent or better stability than the original HOE 21 PH can be formulated. In this context, it is useful to recount a brief history. Insulin manufactured by Hoechst during the time period 1986-1990 was very stable. When environmental regulations forced the manufacturing operation to change from chloroform purification to other methods, the insulin stability deteriorated and was actually reduced from years to weeks. Moreover, it was observed that stability could even radically change from batch to batch. This difference in stability is understood by the present inventors to be due to the difference in the impurity profile. The impurities in the originally stable insulin were likely residues of processing that are not soluble in chloroform, and were likely thus never removed. Modem purified insulin has less impurities.

One insulin impurity may be desamido insulin. This impurity is generally not found in modern purified insulins. In embodiments, desamido insulin stabilizes the insulin dimer by forming a hetero-dimer with human insulin.

By way of explanation, it is noted at this juncture that human insulin consists of two peptide chains: an A chain, containing 21 amino acids, and a B chain, containing 30 amino acids. The A and B chains are connected by two disulfide bonds. The two main degradation products of human insulin are desamido insulin, which is generally desamidated at positions A21 or B3 (referring to the A and B chains, respectively). While A21 desamido insulin has a greater ability to stabilize human insulin (because the A chain is more involved in the dimerization reaction), in alternate exemplary embodiments, B3 desamido insulin may also stabilize human insulin. The primary stabilizing mechanism is that desamido insulin forms heterodimers with human insulin, and this keeps the insulin in the dimerized form longer, thus allowing it to stay stable for longer times in the reservoir of an implantable pump.

It is noted that insulin manufactured after the above described change in the purification process in the early 1990's is only stable for 6 weeks. The following presents the impurities and the human insulin formulations that are important for physical and chemical stability.

Desired Attributes for Insulin Used in New Generation Implantable Devices

It is first useful to specify the desired attributes for both intraperitoneal implantable pumps and insulin to be used in such implantable pumps.
1. Suitable for concentrations up to U1000;
2. Physically stable for 3-6 months as agitated in a pump reservoir and passing through an active pumping mechanism. There should be no aggregation of insulin that would either: (a) form deposits on the components of the pump mechanism that would interfere with pump operation, or (b) form deposits that would stimulate a tissue reaction or lead to the build-up of insulin antibodies.
3. Thermally stable at 25° C. storage temperature for two years and then stable at 37° C. in a titanium reservoir for 3-6 months (under the conditions described in (2) above).

It is noted that currently there is only one insulin formulation which may be used for intraperitoneal delivery, namely Sanofi Insuman™ U400 human regular insulin. Sanofi U400 insulin is at its limit of solubility at 400 u/ml. As noted above, it is stable for only 6 weeks in a pump's reservoir, and its shelf life (assuming it is refrigerated) is less than 1 year. Obviously, Insuman™ U400 does not meet the above criteria for new generation implantable pumps that are contemplated in accordance with various embodiments, and would not be useable in such next generation devices. As noted above, in embodiments, desamido insulin may stabilize the insulin dimer by forming a hetero-dimer with human insulin. In embodiments, the use of desamido insulin may also insure that aggregates are not formed, which means that insulins according to various embodiments herein will not trigger antibody formation in the IPS, for example.

Both insulin stability, and its property of not forming aggregates, are intimately connected to preventing the creation of improperly folded insulin monomers. Improperly folded insulin monomers are auto-catalytic with insulin to create more aggregates. This improperly folded monomer happens because in solution there is an equilibrium between monomers and dimers and hexamers. There is always a bit of monomer. Sometimes, however, the monomer adheres to the surface of the reservoir, and the monomer unfolds. When that unfolded monomer, which is now denatured, falls off of the surface because of agitation or sheer force, it then can grab other insulin molecules and unfold them, which then leads to aggregation.

In embodiments, A-21 desamido insulin may be used to scavenge insulin monomers, thus preventing the aggregation process described above. In embodiments the A-21 desamido influence, because of its charge difference will be a stronger (more stable) heterodimer. A dimer between insulin and A-21 is bound more strongly because of the charge interaction than the insulin-insulin homodimer is bound. In, for example, A21 desamido insulin, The A21 asparagine changes to aspartic acid, and the charge changes from negative to positive.

Thus, A-21 desamido insulin may be used to essentially scavenge insulin monomers. As noted, in embodiments, because of its charge difference, the desamido insulin makes for a stronger heterodimer. A heterodimer comprising regular insulin and either A21 or B3 desamido insulin is bound more strongly together because of the charge interaction than that of an insulin-insulin homodimer. This is, as noted, because the desamido insulin has a positive charge on A21, and regular human insulin has the normal negative charge, so the two monomers attract. As regards a regular insulin homodimer, because the dimerized state has a lower free energy, it is more stable. However, due to each monomer having the same negative charge, and the concomitant electrostatic repulsion, the two regular insulin monomers become close enough to dimerize (i.e., homo-dimerize) less often.

In embodiments, a variety of non-insulin molecules may be added back to purified insulin to stabilize it in this way (i.e., hetero-dimerization). In embodiments, A21 Humalog may be added, or A21 Novolog, for example, and similarly, their respective B3 modified versions as well.

In embodiments, the A21 desamido additive concentration can be optimized by using association constants for the hetero-dimer and knowing the proportion of dimer vs hexamer in U400 insulin. Thus, in embodiments, stabilized insulin compositions suitable for use in implantable artificial pancreatic devices can be provided.

In embodiments, purified insulin may be made stronger and more stable by introducing an insulin analog that will form a hetero dimer with the insulin. Such an insulin analog may, as noted, include A21 desamido insulin, B3 desamido insulin, or other insulin analogs. For example, in embodiments, a quantity of between ½ to 10% A21 desamido insulin may be added to a purified insulin preparation, resulting in a more stable insulin.

As noted above, insulin aggregation is driven by the availability of unfolded human insulin monomer which is available to become aggregate. Human insulin monomer concentration is set by the disassociation coefficient for dimer insulin. Due to the charge on the human insulin monomer there is a high disassociation constant for human insulin dimer. A21 desamido insulin monomer is positively charged, as is B3 desamido insulin. For the same reason, desamido homodimer insulin tends to disassociate to monomer. However the heterodimer composed of human insulin and desamido insulin, such as A21 desamido insulin for example, is more stable than either of the homodimers because the negative charge on regular insulin is attracted to the positive charge on the desamido insulin.

The criteria for choosing the optimum percentage of desamido insulin to insulin are that the desamido addition should complex with the greatest possible percentage of available unfolded insulin monomer. If too little desamido is added, there will be a significant amount of available human insulin monomer available for the autocatalytic aggregation process. If too much desamido insulin is added, there will be an excess of unfolded desamido monomer which will lead to aggregation of desamido insulin. Analytical modeling for finding the optimum concentration is not sufficient because of a variety of secondary effects, however based on the known dimer-monomer disassociation coefficient, the range can be reasonably expected to be in the 0.5 to 10% range, and likely in the 5-10% range. Thus, the actual optimum percentage of desamido must be determined by experiment.

It is further noted that the same logic will guide the optimal percentage addition of B3 desamido, as well as any of the other insulin impurities (degraded insulins) which have the capability of lowering the availability of unfolded monomer insulin.

As may be gathered from the above discussion, modern purification methods leave a very clean insulin. Leaving a pure insulin that has almost no impurities means an insulin with no possibility for hetero-dimerization—all that can be made to scavenge monomers, as described above, is the homodimer, which, as noted, has a high dissociation constant, and thus the pure insulin is inherently less stable.

Stabilization by the Use of Insulin Mixtures

In embodiments, a mixture of two insulins—e.g. Human Insulin and Lispro—can be used to destabilize the first step in the insulin denaturation process which takes place in the insulin dimer. It is noted that the insulin dimer is a small percentage (approximately 5%) of the insulin in a vial and the addition of even a small amount of a second insulin is sufficient to create a hetero-dimer—which is more stable than a homo-dimer. It is noted that the use of these mixtures has not been considered for intraperitoneal implantable pumps thus far.

It is also noted that molecular chaperones can stabilize the folded state of the insulin molecule. One example is alpha-crystallin which binds to the self-association surfaces of the insulin molecule, and thus stabilizes both the dimer and the monomer forms of insulin.

In embodiments, a primary component of such a mixture may be human insulin, to which is added a small percentage of one of the additives described above. In embodiments, such a mixture may be provided in an implantable pump in the IPS.

Methods to Protect the Insulin Molecule from the Aqueous Medium

As is well known, insulin cannot unfold in a non-aqueous medium. It cannot denature until it unfolds. In embodiments, the insulin in a reservoir may thus be insulated from denaturation by protecting it from interacting with water in the solution. In embodiments, this may be performed, for example, using one or more of the following:
a. Liposome encapsulation. Liposomes are in lipid bilayers that are stable until they are infused into the fluid that is present in the peritoneal cavity.
b. Niosome encapsulation: Niosomes are nonionic surfactant vesicles, which protect a molecule from an aqueous environment. They can be made by first coating a particle with a surfactant and then coating with cholesterol as a stabilizing and rigidizing agent.
c. Solid encapsulants: e.g. technospheres. Technospheres can be used to stabilize inhaled insulin. A solid encapsulant can be used to surround and protects the insulin molecule until it is exposed to a pH >7. The technosphere then dissolves and releases the insulin into the body. In this exemplary embodiment, insulin would be encapsulated in solid particles of fumaryl diketopiperazine (FDKP), a known technosphere material. These submicron sized particles can, for example, be suspended in a density matched vehicle such as glycerine which would have a pH of between 4 and 7, and thus, due to the low pH, would be non-corrosive to silicon. Because technospheres dissolve at pH 7, as soon as the technospheres reach the intraperitoneal space, they would dissolve and the insulin would escape into the intraperitoneal space. Technosphere material is a permeation enhancer and so the insulin would permeate the blood vessels rapidly and have a short time to peak. Alternatively, technosphere material could be added to any of the insulins discussed above at pH 7.0+ as a permeation enhancer.
It is here noted that the ability to increase permeation and thereby decrease the time to peak drives a true closed loop artificial pancreas system and device. If another 50% reduction in time to peak can be achieved via the use of a permeation enhancer, in embodiments, blood sugars may be controlled in diabetic patients in real time, precisely as is now done by a healthy pancreas. Thus, as described in greater detail below, currently, without accelerating permeation, using exemplary insulins as described above, it takes about 30 minutes for a discharge of prandial insulin to handle a meal containing 70 grams of carbohydrate, for example a hamburger and a roll. If an additional 50% decrease can be achieved, such that it takes only 15 minutes, the patient can add a milk shake. Although the healthy pancreas discharges insulin immediately, the fact is that pancreatic insulin is not active in the periphery until about 15 minutes have passed. Thus, a 15 minute acting closed loop device has effectively the same control over blood sugar in a diabetic that a healthy pancreas has in a normal individual.
Thus, under such scenarios, a diabetic patient may do whatever they wish (eating wise) within reason, and the artificial pancreatic implant device will prevent their blood sugar from going high. While it may not prevent one from going low in a marathon, for example, it will prevent patients from going low anytime they have any glycogen left in their nostrils.
d. Suspend insulin in polyethyleneglycol and ethanol.

Preservative Free Insulin—Filter Considerations

It has been reported that the antimicrobial preservatives, such as phenol or creosol, that are commonly used in insulin can harm the active surfaces of a glucose sensor. This is a problem if a sensor is placed, or finds its way, near to the insulin delivery depot. This problem exacerbates when multiple glucose sensors are used, such as in the contemplated multi-glucose sensor embodiments being developed by PhysioLogic Devices, the assignee hereof. It has also been reported that there is occasionally the appearance of a mild inflammatory reaction in the intraperitoneal cavity with the chronic infusion of U400 insulin containing phenol. It is noted that phenol is a liquid excipient that may be purchased as Phenol or hydroxybenzene. This phenomenon has been proposed as the root cause of intraperitoneal insulin catheter obstruction.

In embodiments, both problems may be solved with the use of a preservative free insulin. In lieu of an antimicrobial in solution with the insulin, antimicrobial filters 0.2 u or less may be used to prevent the entry of bacteria into the pump during a refill.

Additionally, a second filter can be placed in the fluid path to act as a backup to prevent the entry of any escaped bacteria into the body. Further, in embodiments, the reservoir surfaces may be coated with antibacterial materials such as silver to kill escaped bacteria. In order to safely use preservative free insulin, there would be a need to provide redundancy to prevent bacteria from growing in the reservoir and being delivered to the patient by the pump.

Thus, in embodiments, one may use an inlet filter as a primary safety, at the entry to the reservoir, to prevent introduction of bacteria that may be present on the refill needle tip. For redundancy one may also use a secondary filter at the outlet of the reservoir (the inlet to the pump mechanism) as a second barrier. The inlet filter may be, for example, 0.2 microns which is sufficient to prevent entry by bacteria. It can have a large area, greater than 1 square inch, for flow rates sufficient to fill the reservoir rapidly. For insulin compatibility, it can be a hydrophilic filter such as polysulphone. If the filter became blocked, it is noted that it can be back washed by emptying the reservoir.

In case there is a breach in the inlet filter, the backup filter would protect the user. This filter can, for example, have smaller area and smaller pore size. It is expected to block quickly with bacteria when the reservoir is infected. This would trigger a “no flow” alarm. Thus it would serve as a secondary safety for the user and a method to detect and alarm for an infected reservoir.

Alternatively, in embodiments, phenol may be used as an anti-microbial in the insulin reservoir, and then removed from the insulin after the insulin has entered the catheter. This can be done, for example, by modifying a catheter to contain materials that absorb phenols, such as, for example, silicone rubber. In embodiments, the phenol would thus diffuse into less critical sites such as subcutaneous tissue before it reaches the intraperitoneal tissue in the vicinity of the catheter tip. Another approach is to catalytically decompose the phenol by materials placed in the catheter wall¹ so that by the time the insulin reaches the catheter tip, there is no remaining phenol. ¹ As described in Tanev (1994): Titanium-containing mesoporous molecular sieves for catalytic oxidation of aromatic compounds.

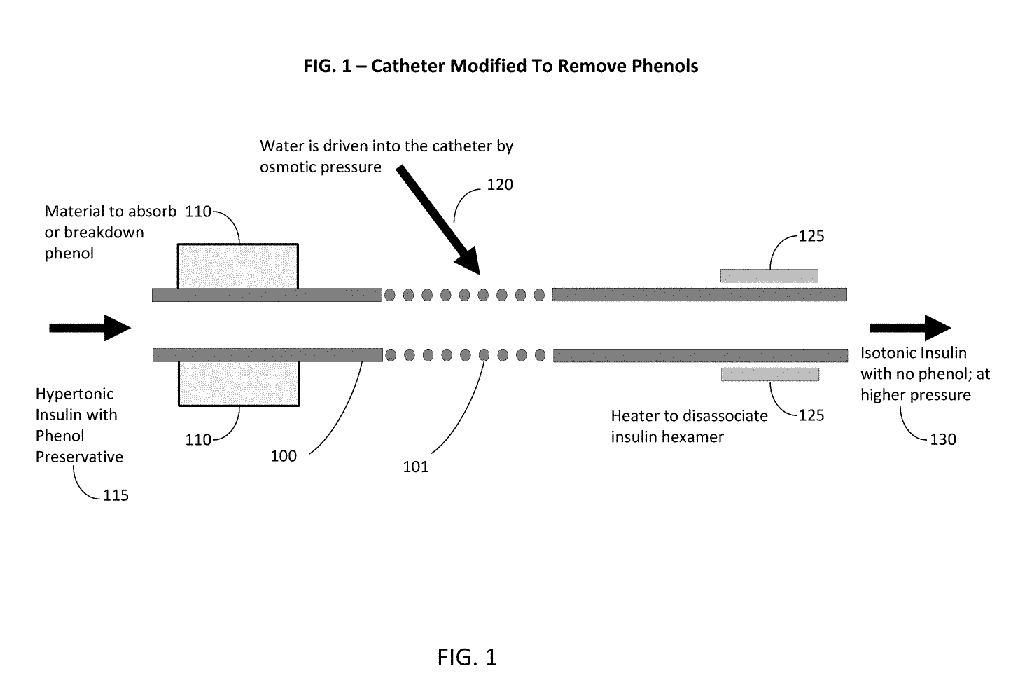

FIG. 1 illustrates this exemplary phenol removal embodiment, according to various embodiments. With reference thereto, hypertonic insulin with a phenol preservative 115 enters catheter 100 from a reservoir (not shown) at the left of the figure. Catheter 100, at its proximal end, comprises a material 110 that can absorb, breakdown, or both absorb and breakdown, phenols. More distally, catheter 100 may have a membrane 101 permeable to water, which is then driven into the catheter by osmotic pressure as shown at arrow 120. At or near the distal end of catheter 100 a heater 125 may be provided (as described below) so as to dissociate the insulin hexamer. As a result, at the exit end of catheter 100, isotonic insulin 130 emerges without phenol, and at a higher pressure; in embodiments, this is what is delivered to the intraperitoneal space.

In alternate embodiments, a given implanted insulin delivery device may be inserted in the upper buttocks site, and in that exemplary case, the catheter to the intraperitoneal space would be somewhat longer.

In embodiments, additives may be added to the insulin so as to increase the osmotic effect. For example, one such additive that will increase the osmotic effect, polyethylene glycol, (PEG) is known to produce an osmotic effect that is greater than would be predicted by molar concentration effects. This means that the molar quantity of PEG added to the insulin would be less than would be required by other excipients to achieve the same osmotic pressure and dilution. It is also well known for its safety in the body because it is a common osmotic laxative. Thus, the use of PEG is a good choice for an osmotic excipient to dilute the catheter lumen contents and apply osmotic pressure to the catheter line.

Ultra Rapid Acting Intraperitoneal Insulin—Removal of Zinc and Phenol Upon Release of the Insulin to Decrease its Time to Peak

It is noted that in order for insulin to diffuse through tissue and capillary walls to reach the blood, insulin must first break down from a stable hexamer form to a dimer, and then to a monomer. This occurs by dilution in body fluids. In embodiments, zinc and phenol (or alternatively, meta-cresol) help stabilize the insulin hexamer. In embodiments, Zinc is added as zinc chloride (ZnCl2). Zn++ is what stabilizes the hexamer. Phenol and meta-cresol are similar molecules used as a bacteriastatic agents. Metacresol is also known as “mcresol.” On the other hand, rapid, repeatable absorption kinetics are the key to real time, physiologic closed loop control of diabetes. The zinc and the phenol (or meta-cresol) are beneficial to physical and chemical stability because they stabilize the insulin hexamer—however this phenomenon slows the breakdown to monomer after the insulin is infused and thus slows the absorption of insulin. So, zinc and phenol (or meta-cresol) are needed initially, but once we wish to inject the insulin into the patient's intraperitoneal space, we want to be rid of them. Thus, removing the phenol (or meta-cresol) as described above may have the additional beneficial effect of destabilizing the insulin hexamer and speeding the breakdown to monomer and uptake of insulin into the tissue. Phenol (or meta-cresol) is also a tissue irritant and aggravates the tendency of tissue to encapsulate and occlude the catheter. If the phenol or meta-cresol were removed from the insulin prior to infusion, then the irritation would be mitigated. As noted, zinc is also present in insulin to stabilize the hexamer configuration. The same beneficial effect would occur if the zinc were removed from the insulin hexamer prior to the insulin reaching the tip of the catheter. This is next described.

It is first noted, however, that the time to peak for conventional subcutaneous insulin may generally be more than one hour, and clearance may be 2 to 3 hours. It is further noted that physiologic insulin is delivered by the pancreas into the blood immediately in response to increased blood sugar (see FIG. 8, G. M. Steil, A. E. Panteleon, K. Rebrin, Closed-loop insulin delivery—the path to physiological glucose control, Advanced Drug Delivery Reviews 56 (2004) 125-144). Because of the lag for conventional insulin injections, or for conventional insulin pumps, a user must inject the insulin before the meal is consumed in order for the insulin action to be synchronous with the meal. The intraperitoneal route, by virtue of the rich capillary beds in both the mesenteric and sphlantic circulatory systems, results in a peak for intraperitoneally discharged regular human insulin in approximately 30 minutes, which is sufficient to control meal excursions using blood glucose measurements alone. In embodiments, this is the threshold for full closed loop insulin delivery. However, it is noted that an excursion for a 70 gram carbohydrate meal could be as much as 200 mg/dl (Lauren M. Huyett, Eyal Dassau, Howard C. Zisser, and Francis J. Doyle, III*, Design and Evaluation of a Robust PID Controller for a Fully Implantable Artificial Pancreas, Industrial & Engineering Chemistry Research (2015)). While this is high, it is still acceptable. It is noted that a meal excursion for a person without diabetes would be less than 120 mg/dl maximum. The present disclosure thus describes four ways for reducing the time to peak for insulin into the bloodstream. In order for insulin to be absorbed through the blood vessel walls into blood, the insulin must disassociate into the monomer form. This is the rate limiting step. In embodiments, four approaches (heat, dilution, zinc removal, and phenol or meta-cresol removal) may be used to speed up the dissociation of the insulin hexamer into dimer so that it can pass through the capillary walls into blood in order to be rapidly absorbed. In embodiments, vibration may further speed up this process by (i) keeping the intraperitoneal fluid “stirred”, thus preventing concentration gradients, and (ii) disturbing the local interstitial fluid and tissue, stimulating fluid motion.

Calculation of Zinc Content for Sanofi U400 Insuman
Assuming that ZnCl₂ is 136.315 g/mol, then there are (0.12/136315)/400 moles of zinc/unit. A mole of zinc is 65,380 mg. Thus a unit of Sanofi U400 insulin contains 0.000144 mg of zinc.
U1000SC is 3 mM. One liter contains 0.003 moles of Zinc. There are 1,000,000 units per liter. There are thus 0.003*65380/1,000,000, or 0.000196 mg of zinc in a unit of U1000SC.

Calculation of the Volume of Zinc in Insulin Delivered Over 20 Years:

@ 55 u/day average use by T1 diabetics:
U1000: 365*20*55*.000196=78.69 mg/7140 mg/cc=0.011 cc Zn
In moles, this is 78.69/65380=0.0012 moles
This would be a deposit 0.117 mm thick over a 3 cm portion of a 1 mm diameter lumen.

Phenol or Meta-Cresol Removal at Catheter

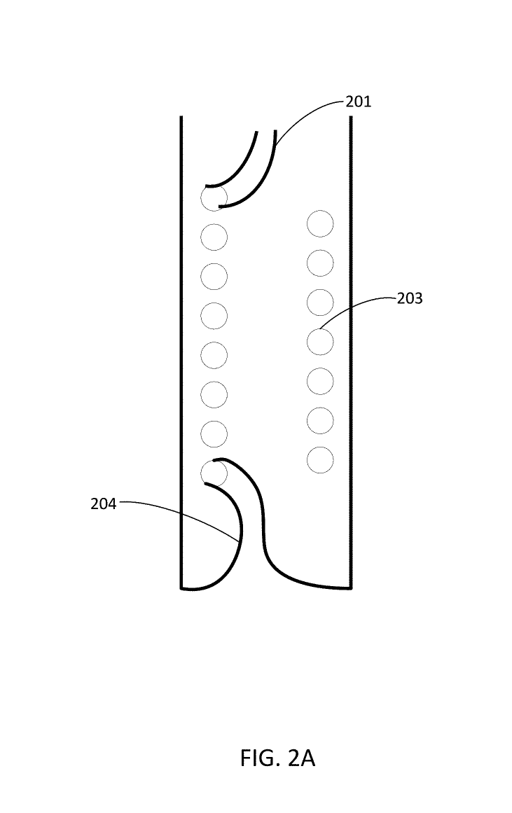

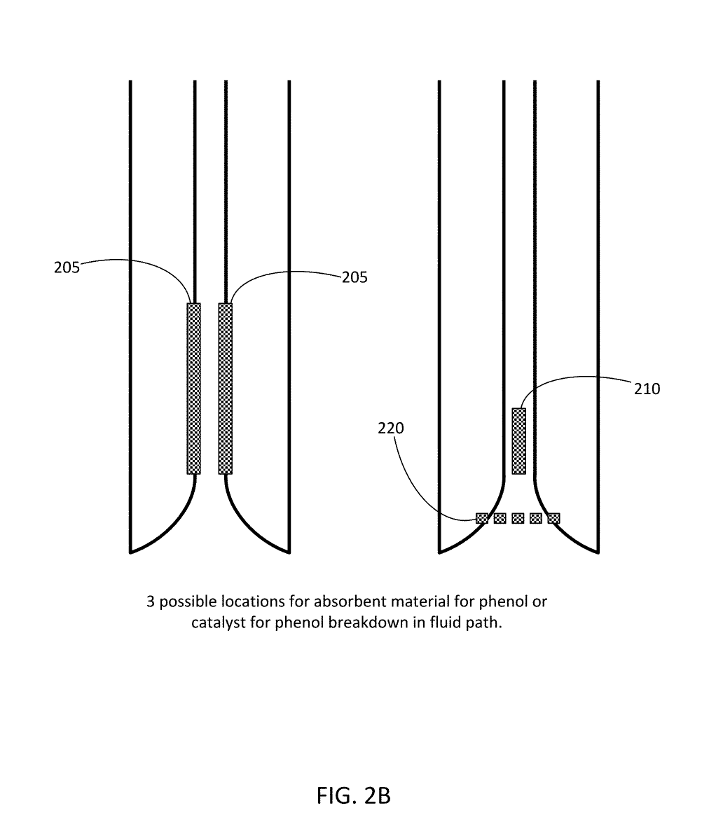

FIGS. 2A and 2B illustrate exemplary methods to remove phenol at the tip of a catheter of an exemplary implantable device, according to various embodiments. With reference to FIG. 2A, to absorb phenol in the silicone rubber used to manufacture the catheter, one may increase the surface to volume ratio at the tip by creating a small diameter spiral portion 203. Thus, the insulin may enter the catheter at 201, run through the spiral portion 203, and exit through an exit portion 204. Or, for example, the surface to volume ratio may be increased by use of a flattened portion of the catheter (minimal cross sectional area), or, for example, a flat duckbill valve tip. Alternatively, in embodiments, one may increase the area of the round lumen by creating a screw thread, or a female spline configuration.

In embodiments, either phenol or a catalyst for phenol may be removed from the insulin passing through the catheter by an absorbent material. FIG. 2B illustrates three possible locations for providing an absorbent material for phenol, or a catalyst for phenol breakdown, in a fluid path. At the edges 205 of the catheter, within the catheter lumen 210, and across the catheter exit 220. It is also noted that the methods shown in FIGS. 2A and 2B may also be used to remove meta-cresol, leading to a similar destabilizing effect and enhanced diffusivity of the insulin.

Zinc Removal at Catheter

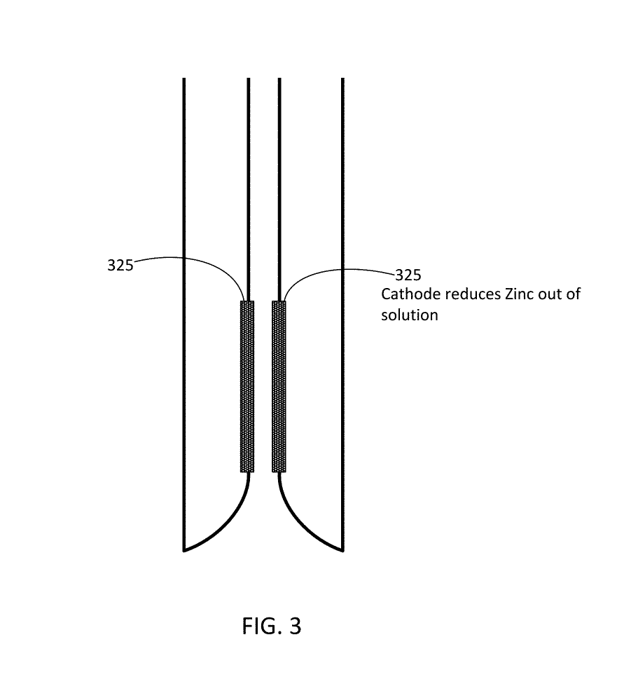

FIG. 3 illustrates exemplary methods to remove zinc at the tip of a catheter of an exemplary implantable device by reduction, according to various embodiments. One approach is to use electrodes 325 in the catheter lumen, such as, for example, those made of electroless palladium, and reduce the zinc as the insulin passes through the electrodes.

Battery Energy Considerations

It is here noted that the battery energy required for the zinc reduction and oxidation according to embodiments as shown in FIG. 3 is:
1 mAh=3.6 C
1 mole=96,485 coulombs
0.0012*96,485=116.13 coulombs
116.13/3.6=32 mAh.

While 32 mAh is small compared to the capacity available in the battery, it is nonetheless significant. Thus, the voltage required for reduction is less than 1 v. This is less than the battery voltage. In embodiments, an optimized circuit design may use this fact to reduce the battery drain by more than one half. It is however noted that, in embodiments, the build-up of zinc may need to be cleared by reversing the current periodically, for example annually. This may double the battery capacity requirement. It is further noted that, in embodiments, there is no need to remove zinc or to speed insulin time to peak during basal rate delivery. This is because during basal rate delivery things are changing slowly, and the time to peak for conventional intraperitoneal insulin is adequate for excellent control. Thus, because basal rate delivery constitutes half of the insulin delivery to a patient, this effect may halve the battery capacity requirement.

Passive Removal of Zinc from an Insulin Solution

An active electrolytic reduction, as described above, consumes energy from the battery in the insulin pump. In embodiments, there are two passive methods for zinc removal that do not use battery energy. These are chelation and passive reduction using a higher negative electrochemical potential material, e.g., manganese and magnesium, which may also be used in accordance with various embodiments.

Passive Reduction Using a Higher Negative Electrochemical Potential Material.

In embodiments, alternatively, another method for removing zinc from insulin as it passes out of a catheter is to place a material in the fluid path with a higher negative electrochemical potential than zinc. Such a material will tend to oxidize and go into solution, and the zinc would be reduced to metallic form and thus be removed from solution. Manganese and magnesium are examples of materials that have a higher negative electrochemical potential than zinc and may thus be used for this purpose, as a metal, in non-oxidized form. They are both endogenous materials and thus will not have deleterious effects in the body in trace quantities.

In embodiments, other materials may also be suitable for passively reducing zinc such as, for example, metal alloys and organic compounds, as long as their negative electrochemical potential is greater than zinc in an aqueous insulin solution. For example, titanium-silver alloys.

Still alternatively, a combination of passive and active reduction of zinc may be used. For example, if a voltage is applied to a passive reduction system, then the rate of reduction of zinc may be increased with reduced energy required from the battery.

Chelation

As is known, chelating agents are materials which bind to metallic ions. They can be complex organic molecules or inorganics materials such as silicates or graphite which are configured physically and chemically to bind to metal ions. Thus, in embodiments, an appropriate chelating agent may be placed in the catheter lumen in contact with insulin.

Configuration

In embodiments, the physical arrangement of the material used to remove zinc by chelating or reducing zinc should maximize surface area. It may be, for example, a helical wire, a lumen coating, a powder or a porous plug.

Removal of Zinc and Phenol in Subcutaneous Systems

It is further noted that the removal of preservative (phenol or meta-cresol) and zinc in the catheter would have the same beneficial effect on sensor survival and insulin delivery kinetics in subcutaneous systems as well as implantable systems, and can thus be implemented in such systems as well, in alternate embodiments.

Heating of the Insulin Upon Release

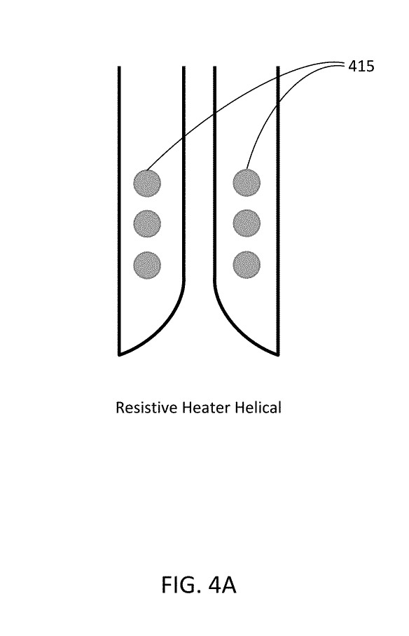

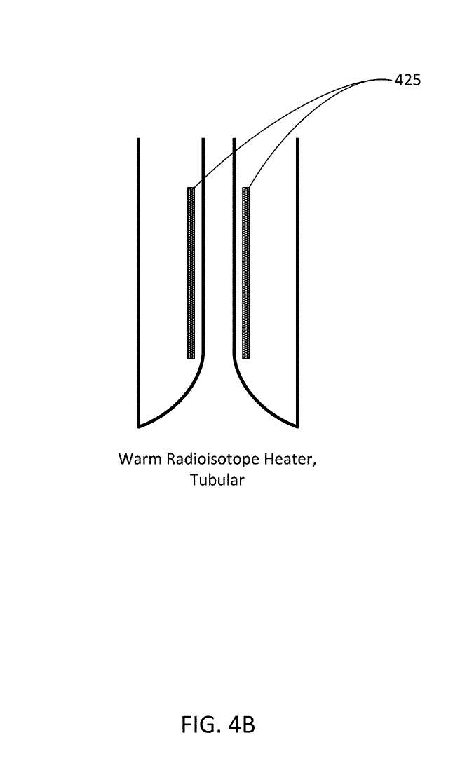

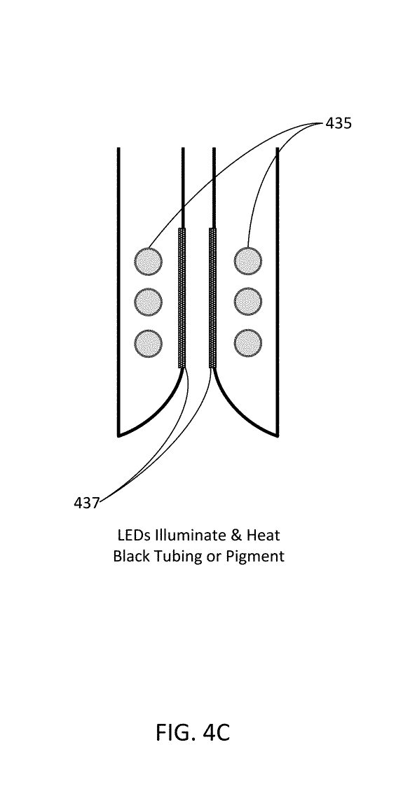

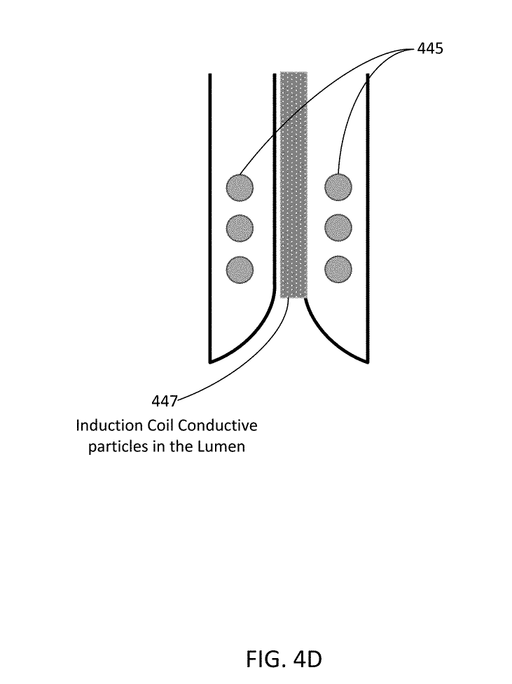

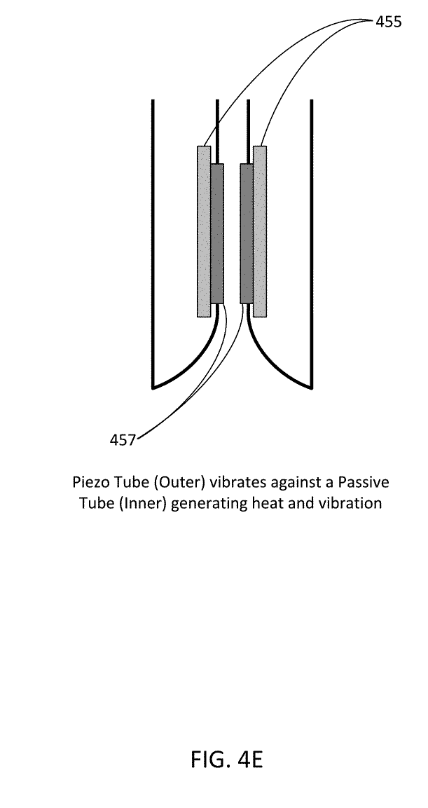

It is further noted that heating insulin has the same effect on speeding the breakdown of hexameric insulin into dimer and monomer, and this effect is leveraged in the heater 125 provided at the distal end of the catheter as shown in FIG. 1. In embodiments, a heat source for such a heater 125 may be provided in various ways, as shown in FIGS. 4A through 4E, which show catheter tip/site configurations for heating and vibrating the insulin and the tissue site, enhancing disassociation and permeability. It is noted that heating insulin to 40° C. accelerates absorption, as per Oliver, Towards a Physiological Prandial Insulin Profile: Enhancement of Subcutaneously Injected Prandial Insulin Using Local Warming Devices (2013).

In general, in embodiments, heat may be generated using a resistive heating element, an exothermic chemical reaction, a photo effect, an inductive heating process or a radioisotope element. Further, it may only be important for prandial insulin (Control of mealtime excursions requires fast insulin. This is not necessary while fasting, for instance while sleeping) thus, the heat source may need only be turned on when there is a need, thus saving energy and battery life.

With reference to FIGS. 4A through 4E, there are shown various exemplary catheter tip/site configurations for heating and vibrating insulin and the tissue site, thereby enhancing disassociation, as well as permeability. These examples generally require power from the battery, and are next described.

As shown in FIG. 4A, there can be a helical resistive heater 415, or, as shown in FIG. 4B, a tubular warm radioisotope heater 425. It is noted in FIGS. 4A through 4E that what is shown is a cross section through a catheter. Thus, any cylindrical or cylindrical structures will be shown as two isolated pieces. Alternatively, as shown in FIG. 4C, LEDs 435 embedded in the catheter may be used to illuminate and heat either a black tubing 437 or a black pigment 437 provided on the inner surface of the lumen. Still alternatively, as shown in FIG. 4D, an induction coil 445 may be provided in the catheter, with conductive particles 447 provided in the lumen. Alternatively (not shown) a wire or coil may be placed directly in the fluid path, and a corresponding inductive coil 445 may be placed in the catheter wall. When current is provided in the inductive coil in the catheter wall, then a current would also be generated in the wire or coil in the fluid path, thus heating the insulin. Finally, as shown in FIG. 4E, a Polyvinylidene fluoride or polyvinylidene difluoride (PVDF) or a piezoceramic tube 455 may be provided in the catheter, that vibrates against a passive tube 457 made from a material suitable for transmitting vibration into fluid, such as, for example, alumina or stainless steel, generating heat and vibration.

Dilution by Osmotic Action

Referring back to FIG. 1, another exemplary method to speed the breakdown of insulin and the entry into tissue would be to dilute the insulin as it is moving through the catheter. If the catheter wall were made from a semi permeable membrane material such as cellulose acetate and the insulin contained more solute (e.g., NaCl) than the surrounding tissue, osmosis would drive water into the catheter lumen, thereby diluting the insulin before it emerged from the catheter tip. Dilution would speed the breakdown of the insulin. It would also have the added advantage of adding osmotic pressure at the tip of the catheter to push out potential obstructions. However, as noted above, in preferred embodiments glycerin can be used to create a hypertonic insulin, and thereby avoid the use of corrosive cations, such as Cl—.

Osmotic Pressure

To appreciate the benefits of the osmotic engine shown in FIG. 1, it is necessary to know the molar concentration of dissolved species in order to calculate the osmotic pressure of an aqueous solution. We calculate the osmotic pressure, π (pi), using the following equation, assuming that glycerin is used to control tonicity:

π=MRT,

where:
M is the molar concentration of dissolved species (units of mol/L);
R is the ideal gas constant (0.08206 L atm mol⁻¹ K⁻¹, or other values depending on the pressure units); and
T is the temperature on the Kelvin scale.

Calculation of Pressure by Doubling Glycerol Concentration in Sanofi U400

Add 16 mg of glycerol/ml to have a total of 32 mg/ml
Glycerol in Sanofi U400

| | |
|---|---|
| 92 | Glycerol mol wt |
| 0.02 | grams/ml |
| 0.17 | molar |
| 0.08 | R |
| 310 | Body temp in Kelvin |
| 4.42 | atm |
| 65 | psid |

Illustrative examples of the technologies disclosed herein are provided below. An embodiment of the technologies may include any one or more, and any combination of, the examples described below.

### EXAMPLES

Example 1 may include an insulin formulation wherein the concentration of OH— in the formulation is such that the pH of the formulation is in the range of about 6.0 to 7.0.

Example 2 may include the insulin formulation of example 1, or other example herein, wherein the pH is less than about 6.4.

Example 3 may include an insulin formulation wherein the concentration of a chlorine ion (cation) in the formulation is reduced by substituting one of a bromine cation or organic materials to maintain isotonicity.

Example 4 may include the insulin formulation of example 3, or other example herein, wherein said organic materials include glycerine.

Example 5 may include an insulin formulation that includes an insulin analog that forms a hetero-dimer with human insulin, in sufficient concentration of the insulin analog so as to complex with the greatest possible percentage of available unfolded insulin monomer.

Example 6 may include the insulin formulation of example 5, or other example herein, wherein the insulin analog is further in sufficient concentration to scavenge insulin monomers, but not in a high enough concentration to create an excess of unfolded monomer of the insulin analog.

Example 7 may include the insulin formulation of example 5, or other example herein, wherein the insulin analog is at least one of: desamido insulin, A21 desamido insulin, B3 desamido insulin, or Lispro.

Example 8 may include the insulin formulation of example 7, or other example herein, wherein the percentage of insulin analog in the formulation is one of: from 0.5% to 10%, from 5% to 10%, or from 1% to 1.5%.

Example 9 may include an insulin formulation that includes a mixture of two different insulins to create a hetero-dimer.

Example 10 may include the insulin formulation of example 8, or other example herein, wherein the formulation contains Lispro and human insulin.

Example 11 may include an insulin formulation that has been at least one of: stabilized with at least one of molecular chaperones or alpha-crystallin; encapsulated with at least one of liposomes, niosomes or technosperes; or encapsulated in fumaryl diketopiperazine (FDKP).

Example 12 may include a closed device for introducing preservative-free insulin into the intraperitoneal space, comprising: a pump; and an insulin reservoir comprising including antimicrobial filters in the tubing leading to the pump, wherein the device is disposed in the intraperitoneal space of a human.

Example 13 may include the device of example 12, or other example herein, further comprising a second backup filter, provided between the antimicrobial fibers and the pump.

Example 14 may include the device of any of examples 12-13, or other example herein, wherein at least one of: the interior surfaces of the reservoir are coated with silver; the insulin is introduced subcutaneously; or the insulin in the reservoir is preserved or stabilized with one of phenol and zinc, but then removed in tubing prior to discharge into the intraperitoneal space.

Example 15 may include the device of example 14, or other example herein, wherein the insulin in the reservoir is stabilized with zinc, and the zinc is removed prior to discharge of the insulin into a body by at least one of (i) reducing the zinc as the insulin passes through electrodes provided in the catheter lumen, (ii) chelation, or (iii) passive reduction using a material having a higher negative electrochemical potential than zinc.

Example 16 may include the device of example 15, or other example herein, wherein said zinc is removed prior to discharge by passive reduction using a material having a higher negative electrochemical potential than zinc.

Example 17 may include the device of example 16, or other example herein, wherein said material is one of manganese or magnesium.

Example 18 may include the device of example 15, or other example herein, wherein said zinc is removed prior to discharge by a combination of active and passive reduction.

Example 19 may include a method of introducing substantially phenol or meta-cresol free insulin into the intraperitoneal space of a human through a catheter, pump and reservoir, comprising: providing a phenol or meta-cresol containing insulin in the reservoir; providing phenol or meta-cresol removing materials in the catheter leading to the pump and reservoir; and discharging the insulin from the reservoir into the catheter such that the phenol or meta-cresol is largely removed as it moves through said catheter.

Example 20 may include the method of example 19, or other example herein, wherein the phenol or meta-cresol removing materials include at least one of absorptive materials, or materials that catalytically decompose the phenol or meta-cresol, as the case may be.

Example 21 may include a method of introducing insulin that breaks down faster into a monomer when introduced into the intraperitoneal space through a reservoir, pump and catheter into the intraperitoneal space, comprising: passing the insulin over phenol absorbing materials in the catheter leading from the pump and reservoir to the intraperitoneal space.

Example 22 may include a method of introducing insulin that breaks down more quickly into a monomer when introduced into the intraperitoneal space through a reservoir, pump and catheter into the intraperitoneal space, comprising: heating the insulin in the catheter leading to the intraperitoneal space.

Example 23 may include the method of example 22, or other example herein, wherein said heating the insulin includes providing in the catheter at least one of: a tubular warm radioisotope heater, an induction coil and corresponding coil or wire in the fluid flow path, or a piezo tube arranged to vibrate against a passive tube.

Example 24 may include an apparatus for introducing insulin that breaks down faster into a monomer when introduced into the intraperitoneal space through a reservoir, pump and catheter into the intraperitoneal space, the improvement comprising including a heating element in the catheter leading to the intraperitoneal space.

Example 25 may include the apparatus of example 24, or other example herein, wherein said heating element includes at least one of: a tubular warm radioisotope heater, an induction coil and corresponding coil or wire in the fluid flow path, or a piezo tube arranged to vibrate against a passive tube.

Example 26 may include a method of introducing insulin that breaks down faster into a monomer when introduced into the intraperitoneal space from a reservoir through a pump and catheter, comprising: providing insulin in the reservoir so that it is hypertonic relative to tissue surrounding the catheter; and passing the hypertonic insulin through catheter walls comprising a semi-permeable membrane material, such that water is driven into the catheter by osmosis so as to dilute the insulin before it emerges from the catheter tip into the intraperitoneal space.

Example 27 may include the method of example 26, or other example herein, wherein the tonicity of said insulin is controlled by adding glycerol.

Example 28 may include the method of example 27, or other example herein, wherein the insulin is Sanofi U400 insulin, and the total glycerol content is made to equal between 16 mg/mol and 32 mg/mol.

Example 29 may include a method of providing osmotic pressure at the tip of a catheter used to discharge insulin via a reservoir, pump and catheter into the intraperitoneal space, comprising: providing a semi-permeable membrane in the catheter; providing insulin in the reservoir so that it is hypertonic relative to tissue surrounding the catheter; and passing the hypertonic insulin through catheter walls comprising a semi permeable membrane material, such that water is driven into the catheter by osmosis so as to generate a defined osmotic pressure at the tip of the catheter, said osmotic pressure pi defined as π=MRT, wherein:

M is the molar concentration of dissolved species (units of mol/L);
R is the ideal gas constant (0.08206 L atm mol−1 K−1, or other values depending on the pressure units); and
T is the temperature on the Kelvin scale.

Example 30 may include the method of example 29, or other example herein, further comprising adding an additive to the insulin to increase the osmotic effect.

Example 31 may include the method of example 30, or other example herein, wherein the additive is polyethylene glycol (PEG).

In accordance with various exemplary embodiments of the present invention, various novel methods for stabilizing insulins, beneficial for use in new generation implantable autonomous insulin pumping devices are presented.

Various citations are referenced throughout the specification (or in footnotes). The disclosures of all citations in the specification are expressly incorporated herein by reference.

While some implementations have been described herein, it should be understood that they have been presented by way of example only, and not limitation. Thus, the breadth and scope of the present application should not be limited by any of the implementations described herein, but should be defined only in accordance with the following and later-submitted claims and their equivalents.

1-11. (canceled)

12. A closed device for introducing preservative-free insulin into the intraperitoneal space, comprising:
a pump; and
an insulin reservoir comprising including antimicrobial filters in the tubing leading to the pump,
wherein the device is disposed in the intraperitoneal space of a human.

13. The device of claim 12, further comprising a second backup filter, provided between the antimicrobial fibers and the pump.

14. The device of claim 12, wherein at least one of:
the interior surfaces of the reservoir are coated with silver;
the insulin is introduced subcutaneously; or
the insulin in the reservoir is preserved or stabilized with one of phenol and zinc, but then removed in tubing prior to discharge into the intraperitoneal space.

15. The device of claim 12, wherein the insulin in the reservoir is stabilized with zinc, and the zinc is removed prior to discharge of the insulin into a body by at least one of (i) reducing the zinc as the insulin passes through electrodes provided in the catheter lumen, (ii) chelation, or (iii) passive reduction using a material having a higher negative electrochemical potential than zinc.

16. The device of claim 15, wherein at least one of:
the zinc is removed prior to discharge by passive reduction using a material having a higher negative electrochemical potential than zinc,
the zinc is removed prior to discharge by passive reduction using one of manganese or magnesium: or
the zinc is removed prior to discharge by a combination of active and passive reduction.

17-18. (canceled)

19. A method of introducing substantially phenol or meta-cresol free insulin into the intraperitoneal space of a human through a catheter, pump and reservoir, comprising:
providing a phenol or meta-cresol containing insulin in the reservoir;
providing phenol or meta-cresol removing materials in the catheter leading to the pump and reservoir;
discharging the insulin from the reservoir into the catheter such that the phenol or meta-cresol is largely removed as it moves through said catheter.

20. The method of claim 19, wherein the phenol or meta-cresol removing materials include at least one of absorptive materials, or materials that catalytically decompose the phenol or meta-cresol, as the case may be.

21. An method of introducing insulin that breaks down faster into a monomer when introduced into the intraperitoneal space through a reservoir, pump and catheter into the intraperitoneal space, comprising at least one of:
passing the insulin over phenol absorbing materials in the catheter leading from the pump and reservoir to the intraperitoneal space;
heating the insulin in the catheter leading to the intraperitoneal space;
heating the insulin in the catheter leading to the intraperitoneal space by providing in the catheter at least one of: a tubular warm radioisotope heater, an induction coil and corresponding coil or wire in the fluid flow path, or a piezo tube arranged to vibrate against a passive tube;
providing insulin in the reservoir so that it is hypertonic relative to tissue surrounding the catheter, and passing the hypertonic insulin through catheter walls comprising a semi-permeable membrane material, such that water is driven into the catheter by osmosis so as to dilute the insulin before it emerges from the catheter tin into the intraperitoneal space;
providing insulin in the reservoir so that it is hypertonic relative to tissue surrounding the catheter, and passing the hypertonic insulin through catheter walls comprising a semi-permeable membrane material, such that water is driven into the catheter by osmosis so as to dilute the insulin before it emerges from the catheter tin into the intraperitoneal space, and wherein either:
the tonicity of the insulin is controlled by adding glycerol, or
the tonicity of the insulin is controlled by adding glycerol and the insulin is Sanofi U400 insulin, and the total glycerol content is made to equal between 16 mg/mol and 32 mg/mol.

24-28. (canceled)

29. A method of providing osmotic pressure at the tip of a catheter used to discharge insulin via a reservoir, pump and catheter into the intraperitoneal space, comprising:
providing a semi-permeable membrane in the catheter;
providing insulin in the reservoir so that it is hypertonic relative to tissue surrounding the catheter; and
passing the hypertonic insulin through catheter walls comprising a semi permeable membrane material, such that water is driven into the catheter by osmosis so as to generate a defined osmotic pressure at the tip of the catheter, said osmotic pressure pi defined as π=MRT,
wherein:
M is the molar concentration of dissolved species (units of mol/L);
R is the ideal gas constant (0.08206 L atm mol−1 K−1, or other values depending on the pressure units); and
T is the temperature on the Kelvin scale.

30. The method of claim 29, further comprising adding an additive to the insulin to increase the osmotic effect.

31. The method of claim 30, wherein the additive is polyethylene glycol (PEG).
